(19) United States
(12) Patent Application Publication
(10) Pub. No.: US 2018/0369137 A1
(43) Pub. Date: Dec. 27, 2018

(54) Natural Lubricant

(71) Applicant: LifeStyles Healthcare Pte. Ltd., Singapore (SG)

(72) Inventors: KC Nguyen, Dothan, AL (US); Ji An Chooi, Bayan Lepas (MY); Chayaporn Pongthanomsak, Suratthani (TH); Dudsadee Phedtong, Surat Thani (TH); Sek Yan Tan, Selangor (MY); Maizatul Shuhada Ahmad, Shah Alam (MY)

(21) Appl. No.: 16/018,697

(22) Filed: Jun. 26, 2018

Related U.S. Application Data
(60) Provisional application No. 62/525,382, filed on Jun. 27, 2017.

(51) Int. Cl. A61K 9/00; A61K 47/36; A61K 47/12

(52) U.S. Cl. CPC A61K 9/0034; A61K 47/12; A61K 47/36

(57) ABSTRACT

Personal lubricant compositions comprise: water, sclerotium gum, carrageenan, and one or more natural emollients. The compositions may comprise by weight: sclerotium gum in an amount in the range of about 0.5% to about 3.0%; carrageenan in an amount in the range of about 0.1% to about 3.0%; and one or more natural emollients in an amount in the range of about 0.5% to about 9.0%; and water. The compositions may be essentially free of one or more of paraben, glycerin or propylene glycol. The personal lubricant compositions may be tailored depending on the application. Sperm-friendly or rectal lubricants may have a pH of about 6 to about 8; and lubricants to promote vaginal health may have a pH of about 4 to about 5.

### CROSS-REFERENCE TO RELATED APPLICATIONS

This application claims priority under 35 U.S.C. § 119(e) to U.S. Provisional Application No. 62/525,382, filed Jun. 27, 2017, the disclosure of which is incorporated herein by reference in its entirety.

### FIELD

The present disclosure relates generally to personal lubricants. Personal lubricants herein are designed with natural ingredients, and may be formulated for vaginal or rectal use. The personal lubricants also maintain a healthy environment for sperm motility.

### BACKGROUND

Personal lubricants can be formulated for various applications, including treating vaginal dryness, providing lubricity and comfort during intimate relations, and lubricating medical devices for insertion. Personal lubricants are formulated to be easy to apply and handle while being effective in providing lubricity and comfort. Some personal lubricants can be irritating to users. There is a continuing need to formulate personal lubricants that are non-irritating to users while maintaining excellent lubricity and comfort.

### SUMMARY

Described herein are personal lubricant formulations, and methods of use thereof. The personal lubricant formulations herein are made from ingredients found in nature and well-accepted among health-conscious consumers. At the same time, performance that substantially matches conventional lubricant by one or more measures is unexpected with these materials. Various advantages, aspects, and features of the present disclosure, as well as details of illustrated embodiments thereof, will be more fully understood from the following description.

In an aspect, a personal lubricant composition comprises: water, sclerotium gum, carrageenan, and one or more natural emollients. The personal lubricant may optionally comprise lactic acid. The composition may comprise by weight: the sclerotium gum in an amount in the range of about 0.5% to about 3.0%; the carrageenan in an amount in the range of about 0.1% to about 3.0%; the one or more natural emollients in an amount in the range of about 0.5% to about 9.0%; and water. The composition may further comprise by weight: lactic acid in an amount in the range of about 0.001% to about 0.1%.

The foregoing summary is not intended, and should not be contemplated, to describe each embodiment or every implementation of the present invention. The Detailed Description and exemplary embodiments therein more particularly exemplify the present invention.

### DETAILED DESCRIPTION

Personal lubricant compositions herein are water-based. Natural gelling agents sclerotium gum (e.g., 0.5 to 3.0 weight %) and carrageenan (e.g., 0.1 to 3.0 weight %) are added to water to provide a soft and supple touch. Both ingredients are synthesized from natural sources and the resulting composition has an aesthetic and feel that duplicates natural vaginal secretions. For added moisture properties, propanediol (0.5 weight %-9.0 weight %) can be added to the formulation as a natural replacement of synthetic glycols. Propanediol provides improved sensorial perceptions relative to synthetic glycols, which gives a smooth feeling leave the skin feeling moisturized. Aloe vera or aloe vera extract (<=2.0 weight %), which is a natural emollient, may also be included to provide soothing effect for the user. Lactic acid (<=2.0%) is naturally secreted by Lactobacillus in the vagina and may be added to the composition to maintain healthy vaginal environment, to ensure that the optimum pH of vagina is maintained during usage. Optionally, antioxidant agents, sodium levulinate (<=2.0 weight %) and/or sodium benzoate (<=2.0 weight %) may be added to impede or prevent microbial activity. The compositions made herein yield a translucent gel of pH range from 3.0-8.0 and viscosity of 1000 cps-10,000 cps. The osmolality of the composition ranges from 250 mOsm/kg to 1500 mOsm/kg.

Reference to “natural” herein means an ingredient occurring in nature and is obtained from a renewable resource. In one or more embodiments, the natural ingredients herein are plant-based.

All percentages herein, unless otherwise specified, are weight percentage of the composition.

Methods of making the personal lubricant compositions include heating water to a temperature in the range of 60 to 90° C. and adding gelling agents sclerotium gum and carrageenan and mixing. Depending on the application, further ingredients such as natural emollients, pH adjusters, and antioxidant agents may be added by mixing.

In a detailed embodiment, provided is a lubricant comprising by weight in water sclerotium gum in an amount in the range of about 0.5% to about 3.0%; the carrageenan in an amount in the range of about 0.1% to about 3.0%; the one or more natural emollients in an amount in the range of about 0.5% to about 9.0%.

In embodiments, other than water, no more than about 3.0% of non-enumerated ingredients (for any given embodiment) are present. In embodiments, other than water, no more than about 2.0% of non-enumerated ingredients are present. In embodiments, other than water, no more than about 1.0% of non-enumerated ingredients are present.

In embodiments, the non-water ingredients are about 98% or more natural ingredients. In embodiments, the non-water ingredients are about 99% or more natural ingredients. In embodiments, the lubricant is essentially free (no more than about 50 ppm) of one or more of paraben, glycerin or propylene glycol.

The natural emollient can be for example propanediol (propane-1,3-diol) or any water-soluble natural extract that provides the sensation to the uses of moisturizing, soothing or softening the skin. Examples include aloe vera and/or extracts from aloe plants (aloe vera-derived ingredients), especially aloe vera extracts. Additional examples include chamomile, and the like. Other emollient examples include for example phenethyl alcohol, undecyl alcohol, and the like. In one or more embodiments, extract emollients may contribute to about 0.5% to about 2.0% of the formulation.

Natural antioxidants can be used, such as about 0.2% to about 2.0% benzoic acid.

In some embodiments, the composition is effective as a vaginal lubricant. Lactic acid may be present in an amount from about 0.001% to about 0.1%. The lactic acid can be used to help maintain a pH of about 3.5 to about 5.5. Lactic acid further matches the lactic acid found in the vagina and helpful to vaginal health. In one or more embodiments, a vaginal lubricant contains 100% natural ingredients.

In some embodiments, the composition is effective as a sperm-friendly lubricant or as a rectal lubricant. For sperm-friendly environments, the composition may have a pH of about 6 to about 8. For rectal use, the composition may also have a pH of about 6 to about 8, which is to suit rectal tissue.

A cushioning effect can be measured by the coefficient of friction together with a consumer survey relative to Durex Love Sex Naturals Intimate Gel. While a cushioning effect is of particular importance for a rectal lubricant, it can be useful in all the lubricant embodiments.

Durex Love Sex Naturals Intimate Gel is said to contain natural ingredients including pre-biotics. The pre-biotic is believed to be marketed as “Bioline,” “a prebiotic which helps maintain the balance of the intimate area.” Ingredients include Aqua [deionized water], glycerin, propanediol, xanthan gum, inulin, benzoic acid, alpha-glucan oligosaccharide, potassium lactate and lactic acid. Other properties are believed to be: pH 3.93, viscosity 2900 cps (spindle 4, speed 60), osmolality 1038.33 mOsm/kg, color slightly yellow, odor slightly sour, appearance clear Gel.

In embodiments, the lubricant measure is lubricity. This can be measured by the coefficient of friction.

In embodiments, the lubricant measure is an aesthetic look that substantially matches healthy natural vaginal secretions. This can be measured by a consumer survey relative to Durex Love Sex Naturals Intimate Gel.

In embodiments, the lubricant measure is its soothing effect on the user. This can be measured physical tests of color and viscosity, and optionally by a blinded survey of appearance relative to Durex Love Sex Naturals Intimate Gel.

In embodiments, the lubricant measure is its lack of harm to, or promotion of, vaginal health. This can be measured by examination of ingredients, pH and osmolality, and optionally by testing relative to Durex Love Sex Naturals Intimate Gel.

In embodiments, the lubricant measure is its lack of damaging effect on natural rubber condoms. In embodiments, the lubricant measure is its lack of damaging effect on synthetic polyisoprene condoms. In embodiments, the lubricant measure is its lack of damaging effect on polyurethane condoms. This lack of damage can be measured by ASTM D7661-10 Standard Test Method for Determining Compatibility of Personal Lubricants with Condoms, such as testing relative to Durex Love Sex Naturals Intimate Gel.

In embodiments, the lubricant is a translucent gel. The pH of the lubricant can be in the range of about 3 to about 8. For a vaginal lubricant, the pH can favorably be about 3 to about 5, such as about 3.5 to about 4.5, such as about 4. For the sperm-friendly embodiment, the pH can favorably be about 6 to about 8, such as about 6.5 to about 7.5, such as about 7. For a rectal lubricant, the pH can favorably be about 6 to about 8, such as about 6.5 to about 7.5, such as about 7.

The osmolality of the lubricant can be about 250 mOsm/kg about 1500 mOsm/kg. For the vaginal lubricant, the osmolarity can favorably be about 400 mOsm/kg to about 1200 mOsm/kg (typically less). For the sperm-friendly embodiment, the osmolarity can favorably be about 300 mOsm/kg to about 600 mOsm/kg, such as about 350 mOsm/kg to about 450 mOsm/kg, such as about 400 mOsm/kg. For the rectal lubricant, the osmolarity can favorably be about 300 mOsm/kg to about 600 mOsm/kg, such as about 350 mOsm/kg to about 450 mOsm/kg, such as about 400 mOsm/kg. Osmolality may be measured in accordance with United States Pharmacopeia (USP)<785>.

The viscosity of the lubricant at 25° C. can be about 1000 cps about 10,000 cps. For the vaginal lubricant, the viscosity can favorably be about 100 cps to less than 10,000 cps. For the sperm-friendly embodiment, the viscosity can favorably be about 1,000 cps to about 5,000 cps. For the rectal lubricant, the viscosity can favorably be about 1,000 cps to about 10,000 cps. Viscosity may be measured in accordance with United States Pharmacopeia (USP)<912>, rotational method using, for example, a Brookfield Viscometer (DV2T LVT), at a testing temperature of 25° C.

Table A provides a summary of exemplary formulations:

TABLE A

| | | A-1 | A-2 |
|---|---|---|---|
| Ingredient | Function | weight % | weight % |
| Sclerotium Gum | emulsion stabilizing/ | 0.5-3 | 0.5-2 |
| | skin conditioning/ | | |
| | viscosity controlling | | |
| Carrageenan | thickener (gelling agent) | 0.1-3 | 0.3-0.6 |
| (Chondrus Crispus) | | | |
| Propanediol | solvent/viscosity | 0.5-9 | 2-9 |
| | controlling/emollient | | |
| Sodium Lactate | buffering/humectant/ | 0-0.5 | 0-0.5 |
| | keratolytic | | |
| Benzoic Acid | bulking/masking/ | 0-2 | 0-0.5 |
| | preservative | | |
| Phenoxyethanol | preservative | 0-1 | 0-1 |
| Lactic Acid | buffering/humectant/ | 0.001-2 | 0.001-0.010 |
| | skin conditioning | | |
| DI Water | Solvent | To total | To total |
| | | 100% | 100% |

Specific embodiments according to the methods of the present invention will now be described in the following examples. The examples are illustrative only, and are not intended to limit the remainder of the disclosure in any way.

All ranges recited herein include ranges therebetween, and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4 or more, or 3.1 or more. If there are two ranges mentioned, such as about 1 to 10 and about 2 to 5, those of skill will recognize that the implied ranges of 1 to 5 and 2 to 10 are within the invention.

### EXAMPLES

### Example 1

A personal lubricant effective as a vaginal lubricant according to the formula of Table 1 was prepared by heating the water to a temperature in the range of 60 to 90° C. and adding the gelling agents: sclerotium gum and carrageenan, and mixing. The further ingredients propanediol, sodium lactate, benzoic acid, and lactic acid were added by mixing.

TABLE 1

| | Vaginal |
|---|---|
| Ingredient | weight % |
| Sclerotium Gum | 1.0000 |
| [Granulated Amigel ®] | |
| Carrageenan (Chondrus Crispus) | 0.4000 |
| [Genuvisco Carrageenan CG-129] | |
| Propanediol (1,3-Propanediol) | 8.0000 |
| [Activonol-3] | |
| Sodium Lactate | 0.4000 |
| [Banggiflow SL 60] | |
| Benzoic Acid | 0.3000 |
| [Benzoic Acid natural] | |
| Lactic Acid | 0.0010 |
| [PURAC ®UltraPure 90] | |
| DI Water | 89.8990 |

The formulation of Table 1 passed 6 month long-term stability testing in accordance with ASTM F 1980—02: Standard Guide for Accelerated Aging of Sterile Medical Device Packages and WHO guideline—WHO Technical Report Series, No. 953, 2009: Annex 2 Stability testing of active pharmaceutical ingredients and finished pharmaceutical products.

### Example 2

A personal lubricant effective as a rectal lubricant according to the formula of Table 2 was prepared by heating the water to a temperature in the range of 60 to 90° C. and adding the gelling agents: sclerotium gum and carrageenan, and mixing. The further ingredients propanediol, benzoic acid, phenoxyethanol, and lactic acid were added by mixing.

TABLE 2

| | Rectal |
|---|---|
| Ingredient | weight % |
| Sclerotium Gum | 1.5000 |
| [Granulated Amigel ®] | |
| Carrageenan (Chondrus Crispus) | 0.5000 |
| [Genuvisco Carrageenan CG-129] | |
| Propanediol (1,3-Propanediol) | 3.0000 |
| [Activonol-3] | |
| Phenoxyethanol | 0.7000 |
| [Phenoxetol] | |
| Lactic Acid | 0.0050 |
| [PURAC ®UltraPure 90] | |
| DI Water | 94.2950 |

The formulation of Table 2 was tested for 6 month stability in accordance with ASTM F 1980—02 WHO Technical Report Series, No. 953, 2009.

### Example 3

A personal lubricant effective as a sperm-friendly lubricant according to the formula of Table 3 was prepared by heating the water to a temperature in the range of 60 to 90° C. and adding the gelling agents: sclerotium gum and carrageenan, and mixing. The further ingredients propanediol, phenoxyethanol, and lactic acid were added by mixing.

TABLE 3

| | Sperm |
|---|---|
| Ingredient | weight % |
| Sclerotium Gum | 1.0000 |
| [Granulated Amigel ®] | |
| Carrageenan (Chondrus Crispus) | 0.4000 |
| [Genuvisco Carrageenan CG-129] | |
| Propanediol (1,3-Propanediol) | 3.0000 |
| [Activonol-3] | |
| Phenoxyethanol | 0.7000 |
| [Phenoxetol] | |
| Lactic Acid | 0.0030 |
| [PURAC ®UltraPure 90] | |
| DI Water | 94.8970 |

The formulation of Table 3 was tested for 6 month stability in accordance with ASTM F 1980—02 WHO Technical Report Series, No. 953, 2009.

### EMBODIMENTS

### Embodiment 1

A personal lubricant composition comprises: water, sclerotium gum, carrageenan, and one or more natural emollients.

### Embodiment 2

The personal lubricant composition of embodiment 1 comprising by weight: the sclerotium gum in an amount in the range of about 0.5% to about 3.0%; the carrageenan in an amount in the range of about 0.1% to about 3.0%; the one or more natural emollients in an amount in the range of about 0.5% to about 9.0%; and water.

### Embodiment 3

The personal lubricant composition of any of embodiments 1 to 2 comprising a pH of about 6 to about 8, which is effective as a sperm-friendly lubricant or as a rectal lubricant.

### Embodiment 4

The personal lubricant composition of embodiment 3 comprising an osmolarity in the range of about 300 mOsm/kg to about 600 mOsm/kg.

### Embodiment 5

The personal lubricant composition of any of embodiments 1 to 4 comprising a viscosity at 25° C. in the range of about 1,000 cps to about 10,000 cps.

### Embodiment 6

The personal lubricant composition of embodiment 5, wherein the viscosity at 25° C. is in the range of about 1,000 cps to about 5,000 cps.

### Embodiment 7

The personal lubricant composition of any of embodiments 1 to 2 comprising a pH of about 3.5 to 5.5, which is effective as a vaginal lubricant.

### Embodiment 8

The personal lubricant composition of any of embodiments 1 to 2 and 6 to 7 further comprising lactic acid.

### Embodiment 9

The personal lubricant composition of embodiment 8 wherein the lactic acid is present in an amount in the range of about 0.001 weight % to about 0.1 weight %.

### Embodiment 10

The personal lubricant composition of any of embodiments 7 to 9 comprising an osmolarity in the range of about 400 mOsm/kg to about 1,200 mOsm/kg.

### Embodiment 11

The personal lubricant composition of any of embodiments 7 to 10 comprising a viscosity at 25° C. in the range of about 100 cps to less than 10,000 cps.

### Embodiment 12

The personal lubricant composition of any preceding embodiment further comprising one or more of: propanediol, benzoic acid, an aloe vera-derived ingredient, chamomile, phenethyl alcohol, and undecyl.

### Embodiment 13

The personal lubricant composition of any preceding embodiment comprising no more than 50 ppm total of one or more of: paraben, glycerin or propylene glycol.

### Embodiment 14

The personal lubricant composition of any preceding embodiment, wherein the non-water ingredients are 98 weight % or more natural ingredients.

### Embodiment 15

The personal lubricant composition of any preceding embodiment, wherein the non-water ingredients are 99 weight % or more natural ingredients.

### Embodiment 16

personal lubricant composition of any preceding embodiment comprising by weight: (a) about 0.5% to about 3.0% of the sclerotium gum; (b) about 0.1% to about 3.0% of the carrageenan; (c) about 0.5% to about 9% of propanediol; (d) 0% to about 0.5% of sodium lactate; (e) 0% to about 2% of benzoic acid; (f) 0% to about 1% phenoxyethanol; and (g) about 0.001% to about 2% lactic acid; and (h) the water.

### Embodiment 17

The personal lubricant of claim 16, wherein ingredients (a) to (h) total 100%.

Where a sentence states that its subject is found in embodiments, or in certain embodiments, or in the like, it is applicable to any embodiment in which the subject matter can be logically applied.

This invention described herein is of a lubricant and methods of forming or using the same. Although some embodiments have been discussed above, other implementations and applications are also within the scope of the following claims. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims. More specifically, those of skill will recognize that any embodiment described herein that those of skill would recognize could advantageously have a sub-feature of another embodiment, is described as having that subfeature.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

What is claimed is:

1. A personal lubricant composition comprises: water, sclerotium gum, carrageenan, and one or more natural emollients.

2. The personal lubricant composition of claim 1 comprising by weight: the sclerotium gum in an amount in the range of about 0.5% to about 3.0%; the carrageenan in an amount in the range of about 0.1% to about 3.0%; the one or more natural emollients in an amount in the range of about 0.5% to about 9.0%; and water.

3. The personal lubricant composition of claim 1 comprising a pH of about 6 to about 8, which is effective as a sperm-friendly lubricant or as a rectal lubricant.

4. The personal lubricant composition of claim 3 comprising an osmolarity in the range of about 300 mOsm/kg to about 600 mOsm/kg.

5. The personal lubricant composition of claim 3 comprising a viscosity at 25° C. in the range of about 1,000 cps to about 10,000 cps.

6. The personal lubricant composition of claim 5, wherein the viscosity at 25° C. is in the range of about 1,000 cps to about 5,000 cps.

7. The personal lubricant composition of claim 1 comprising a pH of about 3.5 to 5.5, which is effective as a vaginal lubricant.

8. The personal lubricant composition of claim 1 further comprising lactic acid.

9. The personal lubricant composition of claim 2 further comprising lactic acid in an amount in the range of about 0.001 weight % to about 0.1 weight %.

10. The personal lubricant composition of claim 7 comprising an osmolarity in the range of about 400 mOsm/kg to about 1,200 mOsm/kg.

11. The personal lubricant composition of claim 7 comprising a viscosity at 25° C. in the range of about 100 cps to less than 10,000 cps.

12. The personal lubricant composition of claim 1 further comprising one or more of: propanediol, benzoic acid, an aloe vera-derived ingredient, chamomile, phenethyl alcohol, and undecyl.

13. The personal lubricant composition of claim 1 comprising no more than 50 ppm total of one or more of: paraben, glycerin or propylene glycol.

14. The personal lubricant composition of claim 1, wherein the non-water ingredients are 98 weight % or more natural ingredients.

15. The personal lubricant composition of claim 14, wherein the non-water ingredients are 99 weight % or more natural ingredients.

16. The personal lubricant of claim 1 comprising by weight:
(a) about 0.5% to about 3.0% of the sclerotium gum;
(b) about 0.1% to about 3.0% of the carrageenan;
(c) about 0.5% to about 9% of propanediol;
(d) 0% to about 0.5% of sodium lactate;
(e) 0% to about 2% of benzoic acid;
(f) 0% to about 1% phenoxyethanol;
(g) about 0.001% to about 2% lactic acid; and
(h) the water.

17. The personal lubricant of claim 16, wherein ingredients (a) to (h) total 100%.
